(19) United States
(12) Patent Application Publication
(10) Pub. No.: US 2022/0117926 A1
(43) Pub. Date: Apr. 21, 2022

(54) USE OF CHLOROGENIC ACID IN PREPARING MEDICINE OR PHARMACEUTICAL COMPOSITION FOR PREVENTING OR TREATING PAIN

(71) Applicant: SICHUAN JIUZHANG BIOLOGICAL SCIENCE AND TECHNOLOGY CO., LTD., Chengdu, Sichuan (CN)

(72) Inventors: Jie ZHANG, Chengdu, Sichuan (CN); Yazhuo ZHANG, Chengdu, Sichuan (CN); Xun JI, Chengdu, Sichuan (CN); Wang HUANG, Chengdu, Sichuan (CN); Wenbin LI, Chengdu, Sichuan (CN)

(21) Appl. No.: 17/422,429

(22) PCT Filed: Jan. 10, 2020
(86) PCT No.: PCT/CN2020/071345

§ 371 Date: Jul. 12, 2021

(30) Foreign Application Priority Data
Jan. 11, 2019 (CN) 201910029815.5

(51) Int. Cl. A61K 31/216; A61P 25/04

(52) U.S. Cl. CPC A61K 31/216; A61P 25/04

(57) ABSTRACT

Chlorogenic acid is used in the preparation of a medicine for preventing or treating pain. The pain can be cancer pain, skeletal muscle pain and the like. A medicine for preventing or treating pain, the medicine being a pharmaceutical preparation prepared by using chlorogenic acid as an active ingredient and adding pharmaceutically acceptable auxiliary materials. Chlorogenic acid may effectively relieve cancer pain and bone joint pain, and may replace analgesic drugs such as opioids and non-steroidal anti-inflammatory drugs in treatment.

### TECHNICAL FIELD

The present invention relates to the field of pain-related drugs, and in particular to the use of chlorogenic acid in the preparation of drugs or pharmaceutical compositions for preventing or treating pain.

### BACKGROUND ART

Pain is a complex, common clinical symptom and disease that reflects subjective emotional feeling. In 1979, the International Association for the Study of Pain (IASP) defined pain as an unpleasant subjective feeling and emotional experience related to tissue damage or potential tissue damage. In 2016, the definition of pain was updated as: pain is a painful experience caused by actual or potential tissue damage, with sensory, emotional, cognitive, and social levels. In 1985, the American Pain Association proposed that pain is the fifth vital sign following heart rate, blood pressure, pulse, and breathing.

The classification of pain is more complicated. In 1994, the pain was classified by IASP according to the specific characteristics of pain, such as location, duration, sustained intensity, cause of the pain, and the pain caused by system disorders. Clifford Woolf believes that the systematic classification of IASP is not enough to guide research and treatment, and divides pain into nociceptive pain, neuropathic pain, and inflammatory pain. If classified according to duration, pain is divided into acute pain and chronic pain, and generally refers to chronic pain in clinical. In the new ICD classification method, chronic pain is divided into the following 7 categories: 1) chronic primary pain; 2) chronic cancer pain; 3) chronic postoperative pain and post-traumatic pain; 4) neuropathic pain; 5) chronic head and maxillofacial pain; 6) chronic visceral pain; 7) chronic skeletal muscle pain.

Among them, cancer pain is a syndrome caused by a series of different pathophysiological changes, including early inflammatory pain, as well as neuropathic pain caused by sensory and sympathetic nerve damage, visceral pain, bone injury, cell death, bone destruction and bone pain as the course of the disease progresses, and at the same time causing anxiety, fear, cognitive impairment, etc., due to pain and emotional changes. Regarding the treatment of cancer pain, American NCCN “Clinical Guidelines in Oncology” (2018 Edition) emphasizes that pain management should reach 5A goal, namely, optimize analgesia (Analgesia), optimize activities of daily living (Activities), minimize adverse effects (Adverse effects), avoid aberrant drug taking (Aberrant drug taking), and relationship between pain and mood (Affect).

In 2011, the Ministry of Health of China formulated and issued “Cancer Pain Diagnosis and Treatment Standards” (2011 edition), pointing out that cancer pain has various causes, which can be roughly divided into three categories: 1) Tumor-related pain: caused by tumor directly invading and oppressing local tissues, tumor metastasis affecting bone tissue, and the like. 2) Pain associated with tumor treatment: commonly caused by surgery, traumatic examinations, radiotherapy, and treatment with cytotoxic chemotherapy drugs. 3) Non-tumor-related pain: including the pain caused by other comorbidities, complications, and other non-tumor factors.

Cancer pain is a complex pathological process. It is currently speculated that cancer pain is caused by the production and secretion of pain mediators by cancer cells in the tumor microenvironment. At the same time, the chemokines or mediators released by tumor cells can recruit other cells such as nerve cells, lymphocytes, endothelial cells, and fibroblasts, and further secrete mediators, such as tumor necrosis factor α (TNF-α), prostaglandin E (PGE), endothelin (ET), interleukin 1 (IL-1), interleukin 6 (IL-6), epithelial growth factor, transforming growth factor B, platelet-derived growth factor, adenosine triphosphate (ATP), nerve growth factor (NGF), etc. These mediators sensitize or activate specific receptors on primary afferent sensory neurons, and plays a role in the body, leading to the occurrence and maintenance of cancer pain.

Therefore, cancer pain assessment is a reasonable and effective analgesic treatment prerequisite, and its assessment should follow the assessment principles of “routine, quantitative, comprehensive and dynamic”. The treatment of cancer pain should also adopt the principle of comprehensive treatment. According to the patient's conditions and physical state, analgesic treatment means can be used, to continuously and effectively eliminate pain, prevent and control adverse drug reactions, reduce the psychological burden caused by pain and treatment, and improve the life quality of patients to the maximum extent possible.

Chronic skeletal muscle pain refers to persistent or recurrent pain caused by bone, joint, muscle or other related soft tissue diseases, which belongs to nociceptive pain. Bone joint pain includes and is not limited to knee joint pain, ankle joint pain, wrist joint pain, elbow joint pain, shoulder joint pain, patellar joint pain, hip joint pain, femoral joint pain, cervical spine, and lumbar spine pain, etc. There are many causes, such as compressive neuropathic pain or somatic referred pain. The pain included in this part has the following characteristics: containing persistent inflammation caused by infectious, autoimmune, or metabolic causes, such as rheumatoid arthritis and gouty inflammation; as well as structural changes that affect bones, joints, tendons or muscles, such as osteoarthritis, soft tissue injury, strain and so on.

At present, the therapeutic methods of cancer pain include etiological treatment, drug analgesic therapy and non-drug therapy (2011 edition of cancer pain diagnosis and treatment standards). Cancer pain is a kind of complication caused by cancer itself, anti-cancer treatment for cancer patients, such as surgery, radiotherapy, or chemotherapy, may remove or relieve cancer pain. Drug analgesic therapy is a three-step analgesic treatment plan formulated by WHO according to cancer pain. According to the pain degree of patients, the analgesic drugs with different intensity were selected. For mild pain, non-steroidal anti-inflammatory drugs (NSAIDs) can be used; for moderate pain, weak opioids can be selected, that may be used in combination with NSAIDs; for severe pain, strong opioids can be selected, that may be used in combination with NSAIDs. NSAIDs are essential drugs for the treatment of mild cancer pain, including ibuprofen, diclofenac, indomethacin, celecoxib, and paracetamol, whose common adverse reactions are peptic ulcer, gastrointestinal bleeding, platelet dysfunction, kidney function damage, liver function damage, etc. Opioids are the choice drug for the treatment of moderate and severe pain. At present, the short-acting drugs used clinically for moderate to severe cancer pain are immediate-release morphine tablets, and long-acting drugs are morphine sustained-release tablets, oxycodone sustained-release tablets, fentanyl transdermal patches and the like, whose adverse reactions mainly include constipation, nausea, vomiting, lethargy, itching, urinary retention, delirium, cognitive impairment, respiratory depression, etc.

Cancer pain is mostly chronic pain. Although WHO three-step analgesic therapy is used clinically to treat cancer pain, the pain control effect is not very satisfactory. 36-50% of cancer patients still endure various degrees of pain. Moreover, three-step analgesics (opioids and NSAIDs) also have serious adverse reactions. Due to the mental dependence of opioid analgesics, that is, addiction, countries around the world have also introduced corresponding drug management programs to strengthen the management and use of opioids.

The drugs used to treat bone and joint pain are similar to those used to treat cancer pain. Both are acetaminophen, COX-2 inhibitors, NSAIDs and Opioids. The treatment strategy is still WHO's three-step analgesic therapy. Obviously, while receiving treatment, patients will also suffer the toxic side effects of these drugs. Therefore, it is extremely urgent to develop a safe and effective drug for the treatment of pain.

### CONTENT OF THE INVENTION

In order to solve the above problem, the present invention first provides the use of chlorogenic acid in preparing drugs or pharmaceutical compositions for preventing or treating pain.

As the use mentioned above, said pain is cancer pain.

As the use mentioned above, said pain is skeletal muscle pain.

As the use mentioned above, said pain is inflammatory pain.

As the use mentioned above, said pain is neuropathic pain.

As the use mentioned above, said pain is immune pain.

Further, said cancer pain is neuropathic pain.

Further, said cancer pain is nociceptive pain.

Further, said cancer pain is chronic pain.

Further, said skeletal muscle pain is bone joint pain.

More further, said bone joint pain is knee joint pain, ankle joint pain, wrist joint pain, elbow joint pain, shoulder joint pain, patellar joint pain, hip joint pain, femoral joint pain, cervical and/or lumbar pain.

As the use mentioned above, preferably, the prevention or treatment of pain is to reduce mechanical hyperalgesia or radiant hyperalgesia caused by cancer.

As the use mentioned above, the drug or pharmaceutical composition is those inhibiting the expression of tumor necrosis factor α (TNF-α), interleukin 1β (IL-1β), and interleukin 6 (IL-6).

As the use mentioned above, the drug or pharmaceutical composition is those regulating/increasing the expression of 5-hydroxytryptamine (5-HT) and dopamine (DA) caused by pain;

The present invention also provides a drug for prevention or treatment of pain, and the drug is a pharmaceutical preparation obtained by using chlorogenic acid as an active ingredient, with addition of pharmaceutically acceptable excipients.

Preferably, the prevention or treatment of pain is to reduce mechanical hyperalgesia or radiant hyperalgesia caused by cancer.

For the drug mentioned above, in the pharmaceutical preparation, each preparation unit contains 1-3000 mg of chlorogenic acid; and the preparation unit is pill, tablet, packet, small ball, ampul or bottle.

For the drug mentioned above, the human dosage of chlorogenic acid is 1-10 mg/kg in the pharmaceutical preparation.

For the drug mentioned above, the pharmaceutical preparations are oral preparations or injections.

The present invention also provides a pharmaceutical composition for preventing or treating pain, characterized in that it is a pharmaceutical composition containing the pharmaceutical preparation mentioned above.

The inventor accidentally discovered that chlorogenic acid can effectively prevent and treat pain, especially cancer pain as well as bone and joint pain. Therefore, the use of chlorogenic acid provided by the present invention in the preparation of drugs for preventing or treating pain has good industrialization value.

In the case of continuous medication, the drug of the present invention can effectively relieve cancer pain as well as bone and joint pain, without toxic side effects. It can replace analgesic drugs such as opioids and non-steroidal anti-inflammatory drugs, avoid the toxic and side effects produced by long-term use of opioids, improve the life quality of patients, and have good clinical application prospects.

Obviously, based on above content of the present invention, according to the common technical knowledge and the conventional means in the field, without department from above basic technical spirits, other various modifications, alternations, or changes can further be made.

By following specific examples of said embodiments, above content of the present invention is further illustrated. But it should not be construed that the scope of above subject of the present invention is limited to following examples. The techniques realized based on above content of the present invention are all within the scope of the present invention.

### DESCRIPTION OF FIGURES

FIG. 1. The curve diagram of the paw withdrawal threshold of rats due to mechanical hyperalgesia in each experimental group of Experimental Example 3.

FIG. 2. The curve diagram of radiant heat pain threshold of rats in each experimental group of Experimental Example 3.

FIG. 3. Histogram of TNF-α expression in serum of rats in each experimental group of Experimental Example 3.

FIG. 4. Histogram of IL-1β expression in serum of rats in each experimental group of Experimental Example 3.

FIG. 5. Histogram of IL-6 expression in serum of rats in each experimental group of Experimental Example 3.

FIG. 6. Histogram of NE content in brain tissue of rats in each experimental group of Experimental Example 3.

FIG. 7. Histogram of DA content in brain tissue of rats in each experimental group of Experimental Example 3.

FIG. 8. Histogram of 5-HT content in brain tissue of rats in each experimental group of Experimental Example 3.

### EXAMPLES

The starting materials and equipment used in the specific examples of the present invention are all known products and can be obtained by purchasing commercially available products.

### Example 1 the Formula for Oral Preparation of the Present Invention

### 1. Formula 1

Chlorogenic acid 1000 g.
Preparative method: chlorogenic acid was aseptically weighed and subpacked as powders.

### 2. Formula 2

Chlorogenic acid 1000 g, bulking agent 500 g, binding agent 5 g.
Preparative method: chlorogenic acid, bulking agent, and binding agent were weighed according to the formula, granulated, sieved, and subpacked as granules.

### 3. Formula 3

Chlorogenic acid 1000 g, bulking agent 500 g, binding agent 5 g, and lubricant 3 g.
Preparative method: chlorogenic acid, bulking agent, and binding agent were weighed according to the formula, granulated, sieved, and then lubricant was added, followed by pressing, to obtain tablets.

Above bulking agents were one or more of mannitol, lactose, starch, microcrystalline cellulose, and dextrin; the binding agents were sodium carboxymethylcellulose and PVP; the lubricants were magnesium stearate, talcum powder, and micro silica gel.

### Example 2 the Formula for Injection of the Present Invention

### 1. Formula 1

Chlorogenic acid 1000 g.
Preparative method (1): chlorogenic acid was aseptically weighed according to the formula, and aseptically subpacked as powder injection.
Preparative method (2): chlorogenic acid was weighed according to the formula, dissolved in water for injection, filtered, sterilized, freeze-dried, and filled, to obtain freeze-dried powder injection.

### 2. Formula 2

Chlorogenic acid 1000 g, stent agent 2667 g, and antioxidant 67 g.
Preparative method: chlorogenic acid, stent agent, and antioxidant were weighed according to the formula, dissolved in water for injection, filtered, sterilized, filled, and freeze-dried to obtain freeze-dried powder injection.

Said stent agents were mannitol, lactose and glucose; the antioxidants were sodium bisulfite, vitamin, glutathione, and folic acid.

### Example 3 Animal Experiment of Chlorogenic Acid on Preventing or Treating Bone Cancer Pain in Rats

1. Experimental Material

1.1 Animals

SD rats, female, weighing 180-200 g, purchased from Chengdu Dossy Experimental Animal Co., Ltd.

1.2 Cell Lines

Walker rat breast cancer cell lines, purchased from Shanghai Institutes for Biological Sciences, Chinese Academy of Sciences.

1.3 Drugs

Chlorogenic acid, batch No.: 171101, with a content of 99.83%, prepared by Sichuan Jiuzhang Biological Science and Technology Co., LTD.

2. Experimental Method

2.1 Preparation of Cell Suspension

After two SD female rats were intraperitoneally injected with 0.5 mL cancer cells (4×10⁴ cells/μL), ascites was collected on the 7th day, and the cells are rinsed 3 times with sterile 0.01 mol/L PBS to adjust to the concentration of 4×10³ cells/μL for use.

2.2 Model Building

60 SD rats were randomly selected, and after anesthesia, a small 1 cm incision was made in the upper tibial skin. A 10 mL syringe needle was used to puncture and perforate, then a 10 μL syringe was inserted into the bone marrow cavity, and 3 μL cell suspension containing 3×10³ Walker rat breast cancer cells was slowly injected. The pinhole was sealed with bone wax, and the skin was sutured. After surgery, the rats were placed on a 37° C. hot plate for rewarming. After recovery, rats were returned to the cage for further breeding, to obtain the model group. In addition, 8 SD rats were randomly selected, and an equal volume of PBS solution was injected into the marrow cavity of the left upper tibia. The rest of the operations were the same as the model group, to obtain the sham operation group.

From the 10th day after the operation, the rats in the model group with mechanical hyperalgesia and radiant thermal pain were screened out by using pain behavior as the criterion.

Mechanical pain hypersensitivity: continuous pain measurement was performed from the 10th day after the operation, the contact stimulator's fine fibers were allowed to touch with the middle of the affected side of the rat's plantar, and the force can be increased up to 80 g within 5 seconds. The rat's paw withdrawal respond was observed, and the paw withdrawal threshold was recorded. Each animal was tested 5 times, with an interval of 5 min between two tests. The paw withdrawal threshold of the model group was significantly different from that of the sham operation group, indicating that there was mechanical hyperalgesia.

Radiation thermal pain response: the rat was placed on a glass plate, and the affected side of the foot was irradiated with a heat radiator. The plantar side of each rat would be tested 3 times at an interval of 5 min. The response time from exposure to paw withdrawal was recorded, and the time to retract the paw was the pain threshold. The pain threshold of the model group was significantly different compared with that of the sham operation group, indicating that there was a radiation thermal pain response.

2.3 Administration of Animals

The rats that had been successfully modeled were randomly divided into groups, eight rats for each group, that included high-dose chlorogenic acid group (80 mg/kg), middle-dose chlorogenic acid group (40 mg/kg), low-dose chlorogenic acid group (20 mg/kg), model negative group (N.S normal saline), and sham operation group (N.S normal saline). The rats were intraperitoneally injected with drugs for 15 days in a volume of 0.2 mL/10 g, once a day; the model-negative group and the sham operation group were given the same volume of normal saline.

2.4 Detection of Indexes

2.4.1 Pain Behavior

From the second day after administration, the mechanical pain hypersensitivity and radiation thermal pain were investigated before each administration, and the paw withdrawal threshold and the pain threshold were recorded.

2.4.2 Cytokines

After the last administration, blood was collected from the eyeballs of the rats, and the animals in each group were sacrificed by neck removal. The blood samples were allowed to stand at room temperature for 20 minutes and then centrifuged. The supernatant was collected and the contents of TNF-α, IL-1β, and IL-6 in the peripheral blood serum of the mice were determined by ELISA. After the rats were sacrificed, the hypothalamus was quickly separated, homogenized in an ice bath with perchloric acid, and centrifuged. The supernatant was collected, and the contents of NE, DA, and 5-HT were determined by HPLC-EC method (Hou Yanning, Wang Na, et al, Effects of progesterone on morphine-induced conditioned place preference and levels of monoamine transmitters in rat brain. Chinese Journal of Pharmacology, 2006, 22(8), 980-983).

3. Statistical Processing

All data were expressed as x±s. SPSS 11.0 software was used for statistical processing and t test analysis. P<0.05 was considered to be significantly different.

4. Experimental Results

4.1 Pain Behavior

4.1.1 Mechanical Pain Hypersensitivity

In the model group selected after operation, during the administration period, the mechanical paw withdrawal threshold was significantly different compared with the sham operation group (p<0.05), indicating that the tibia implant model was successful.

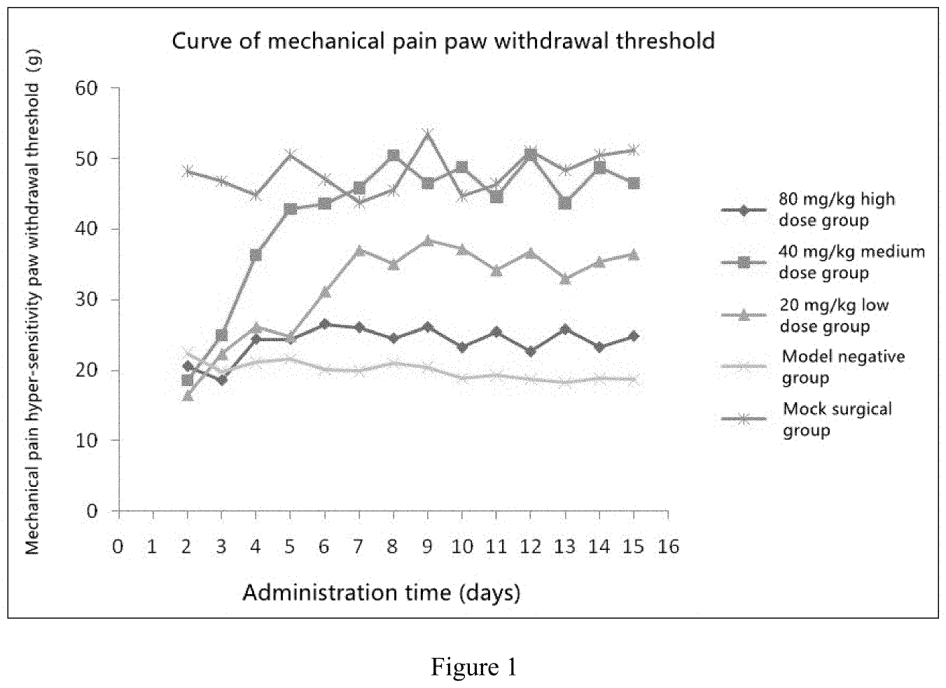

Compared with the model group, paw withdrawal thresholds in 40 mg/kg and 20 mg/kg dose groups of chlorogenic acid had a significant difference from 5-7 days after administration (p<0.05), and especially 40 mg/kg dose group was basically the same as the sham operation group; it showed that the administration dose (20-40 mg/kg) of chlorogenic acid could significantly improve and alleviate the mechanical hyperalgesia caused by bone cancer, that was in a dose-effect relationship. In 80 mg/kg dose group, the mechanical paw withdrawal threshold was different from the model group, but the difference was not statistically significant (p>0.05). The results were shown in Table 1 and FIG. 1.

TABLE 1
The paw withdrawal threshold of mechanical hyperalgesia in rats of each experimental group (x ± s).

| | Dose | | | | | | | | | | | | | | |
|---|---|---|---|---|---|---|---|---|---|---|---|---|---|---|---|
| | (mg/ | Paw withdraw threshold (g) | | | | | | | | | | | | | |
| Groups | kg) | 2 d | 3 d | 4 d | 5 d | 6 d | 7 d | 8 d | 9 d | 10 d | 11 d | 12 d | 13 d | 14 d | 15 d |
| Chloro- | 80 | 20.58 ± | 18.59 ± | 24.41 ± | 24.39 ± | 26.53 ± | 26.07 ± | 24.52 ± | 26.11 ± | 23.21 ± | 25.42 ± | 22.63 ± | 25.79 ± | 23.25 ± | 24.83 ± |
| genic | | 4.73 | 5.14 | 5.26 | 4.15 | 5.19 | 3.15 | 7.01 | 4.88 | 3.45 | 3.74 | 5.23 | 3.42 | 2.45 | 3.89 |
| acid | | | | | | | | | | | | | | | |
| high | | | | | | | | | | | | | | | |
| dose | | | | | | | | | | | | | | | |
| group | | | | | | | | | | | | | | | |
| Chloro- | 40 | 18.53 ± | 24.94 ± | 36.37 ± | 42.83 ± | 43.59 ± | 45.85 ± | 50.41 ± | 46.49 ± | 48.77 ± | 44.59 ± | 50.52 ± | 43.72 ± | 48.73 ± | 46.51 ± |
| genic | | 3.04 | 4.43 | 2.71 | 5.14 | 3.83 | 7.25 | 6.79 | 2.86 | 6.62 | 7.11 | 4.04 | 7.16 | 6.51 | 3.38 |
| acid | | | | | | | | | | | | | | | |
| middle | | | | | | | | | | | | | | | |
| dose | | | | | | | | | | | | | | | |
| group | | | | | | | | | | | | | | | |
| Chloro- | 20 | 16.46 ± | 22.32 ± | 26.14 ± | 24.86 ± | 31.21 ± | 37.06 ± | 35.11 ± | 38.48 ± | 37.23 ± | 34.22 ± | 36.74 ± | 33.02 ± | 35.43 ± | 36.52 ± |
| genic | | 3.73 | 4.15 | 4.11 | 3.51 | 5.82 | 3.45 | 2.21 | 3.76 | 6.82 | 5.38 | 4.28 | 4.41 | 2.42 | 4.05 |
| acid | | | | | | | | | | | | | | | |
| low | | | | | | | | | | | | | | | |
| dose | | | | | | | | | | | | | | | |
| group | | | | | | | | | | | | | | | |
| Model | N.S | 22.42 ± | 19.85 ± | 21.08 ± | 21.63 ± | 20.12 ± | 19.88 ± | 21.04 ± | 20.46 ± | 18.89 ± | 19.27 ± | 18.75 ± | 18.29 ± | 18.82 ± | 18.66 ± |
| negative | | 2.36 | 4.23 | 7.11 | 3.06 | 4.29 | 5.26 | 2.59 | 3.08 | 2.95 | 6.08 | 3.47 | 5.36 | 3.11 | 3.51 |
| group | | | | | | | | | | | | | | | |
| Sham | N.S | 48.23 ± | 46.79 ± | 44.83 ± | 50.52 ± | 47.04 ± | 43.84 ± | 45.52 ± | 53.41 ± | 44.75 ± | 46.42 ± | 51.03 ± | 48.39 ± | 50.52 ± | 51.23 ± |
| oper- | | 7.79 | 5.56 | 9.23 | 8.38 | 3.23 | 6.91 | 4.08 | 6.25 | 8.44 | 6.09 | 3.14 | 4.25 | 4.08 | 5.29 |
| ation | | | | | | | | | | | | | | | |
| group | | | | | | | | | | | | | | | |

Note:
N.S, no drug.

4.1.2 Radiant Heat Pain Response

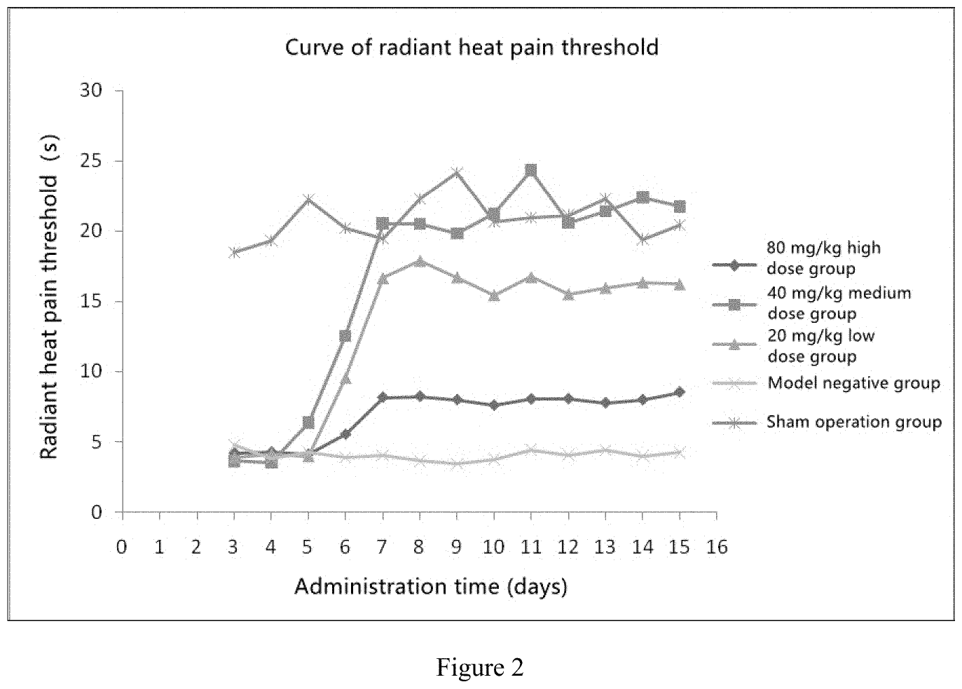

Compared with the model group, radiant heat pain thresholds in 40 mg/kg and 20 mg/kg dose groups of chlorogenic acid had a significant difference from 7 days after administration (p<0.05), and 40 mg/kg dose group was the most significant, and basically equivalent to the sham operation group. It showed that the administration dose (20-40 mg/kg) of chlorogenic acid could significantly improve and alleviate the Radiant heat hyperalgesia caused by bone cancer, that was in a dose-effect relationship. In 80 mg/kg dose group, the mechanical paw withdrawal threshold was different from the model group, but the difference was not statistically significant (p>0.05). The results were shown in Table 2 and FIG. 2.

TABLE 2
The pain threshold of radiant heat pain reaction in rats of each experimental group (x ± s).

| | Dose | | | | | | | | | | | | | | |
|---|---|---|---|---|---|---|---|---|---|---|---|---|---|---|---|
| | (mg/ | Pain threshold (s) | | | | | | | | | | | | | |
| Groups | kg) | 2 d | 3 d | 4 d | 5 d | 6 d | 7 d | 8 d | 9 d | 10 d | 11 d | 12 d | 13 d | 14 d | 15 d |
| Chloro- | 80 | 4.17 ± | 4.24 ± | 4.09 ± | 5.52 ± | 8.15 ± | 8.22 ± | 7.98 ± | 7.59 ± | 8.03 ± | 8.05 ± | 7.76 ± | 7.98 ± | 8.52 ± | 8.38 ± |
| genic | | 2.21 | 2.21 | 1.96 | 3.05 | 2.58 | 3.05 | 2.52 | 3.19 | 3.22 | 1.11 | 2.52 | 3.41 | 2.12 | 3.74 |
| acid | | | | | | | | | | | | | | | |
| high | | | | | | | | | | | | | | | |
| dose | | | | | | | | | | | | | | | |
| group | | | | | | | | | | | | | | | |
| Chloro- | 40 | 3.62 ± | 3.52 ± | 6.36 ± | 12.52 ± | 20.53 ± | 20.49 ± | 19.83 ± | 21.21 ± | 24.32 ± | 20.59 ± | 21.41 ± | 22.39 ± | 21.74 ± | 20.88 ± |
| genic | | 2.43 | 2.42 | 3.41 | 4.01 | 7.41 | 5.72 | 6.09 | 3.64 | 8.41 | 6.77 | 3.62 | 6.11 | 3.98 | 6.43 |
| acid | | | | | | | | | | | | | | | |
| middle | | | | | | | | | | | | | | | |
| dose | | | | | | | | | | | | | | | |
| group | | | | | | | | | | | | | | | |
| Chloro- | 20 | 3.86 ± | 4.11 ± | 3.93 ± | 9.53 ± | 16.66 ± | 17.89 ± | 16.72 ± | 15.45 ± | 16.74 ± | 15.52 ± | 15.97 ± | 16.35 ± | 16.22 ± | 16.75 ± |
| genic | | 2.28 | 3.26 | 4.55 | 5.62 | 4.38 | 3.52 | 4.09 | 2.52 | 5.21 | 2.73 | 3.08 | 4.54 | 2.25 | 1.81 |
| acid | | | | | | | | | | | | | | | |
| low | | | | | | | | | | | | | | | |
| dose | | | | | | | | | | | | | | | |
| group | | | | | | | | | | | | | | | |
| Model | N.S | 4.74 ± | 3.79 ± | 4.21 ± | 3.88 ± | 4.02 ± | 3.62 ± | 3.43 ± | 3.76 ± | 4.42 ± | 4.05 ± | 4.41 ± | 3.98 ± | 4.22 ± | 4.38 ± |
| negative | | 1.28 | 2.00 | 2.05 | 2.51 | 1.63 | 1.56 | 3.13 | 2.93 | 2.26 | 1.88 | 2.92 | 2.06 | 2.19 | 2.77 |
| group | | | | | | | | | | | | | | | |
| Sham | N.S | 18.48 ± | 19.29 ± | 22.21 ± | 20.2 ± | 19.45 ± | 22.27 ± | 24.16 ± | 20.68 ± | 20.93 ± | 21.12 ± | 22.29 ± | 19.41 ± | 20.43 ± | 22.62 ± |
| oper- | | 2.23 | 3.71 | 3.04 | 3.11 | 4.83 | 2.58 | 5.52 | 3.85 | 4.79 | 3.52 | 5.83 | 2.63 | 2.33 | 3.52 |
| ation | | | | | | | | | | | | | | | |
| group | | | | | | | | | | | | | | | |

Note:
N.S, no drug.

4.2 Cytokines

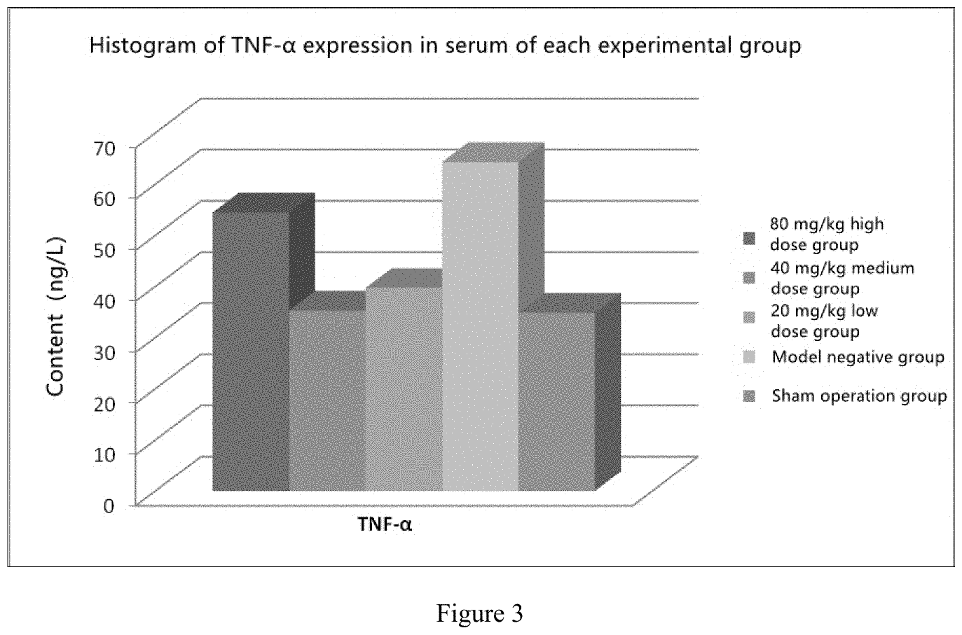

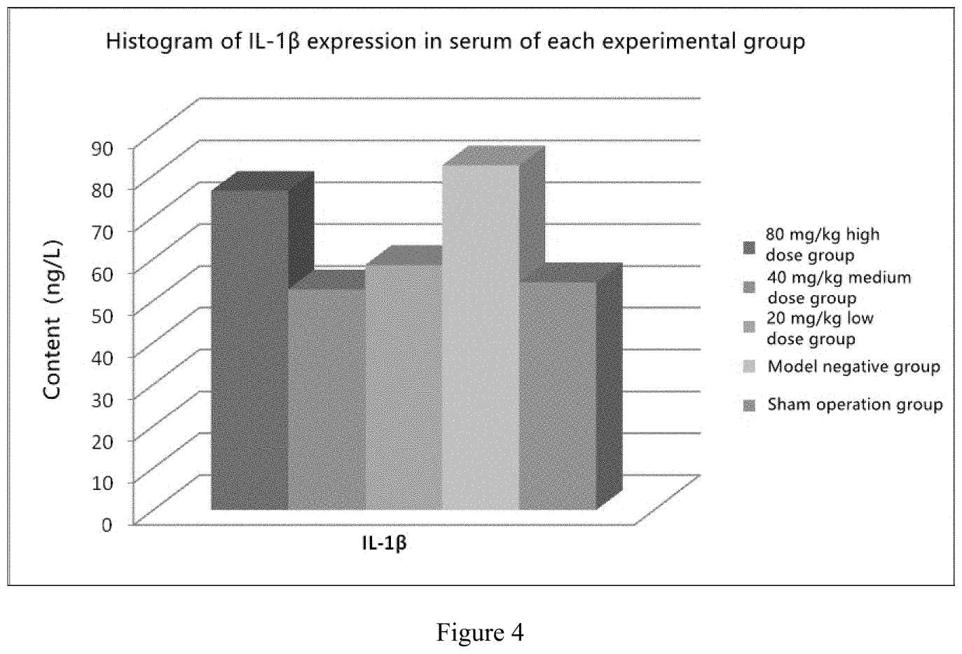

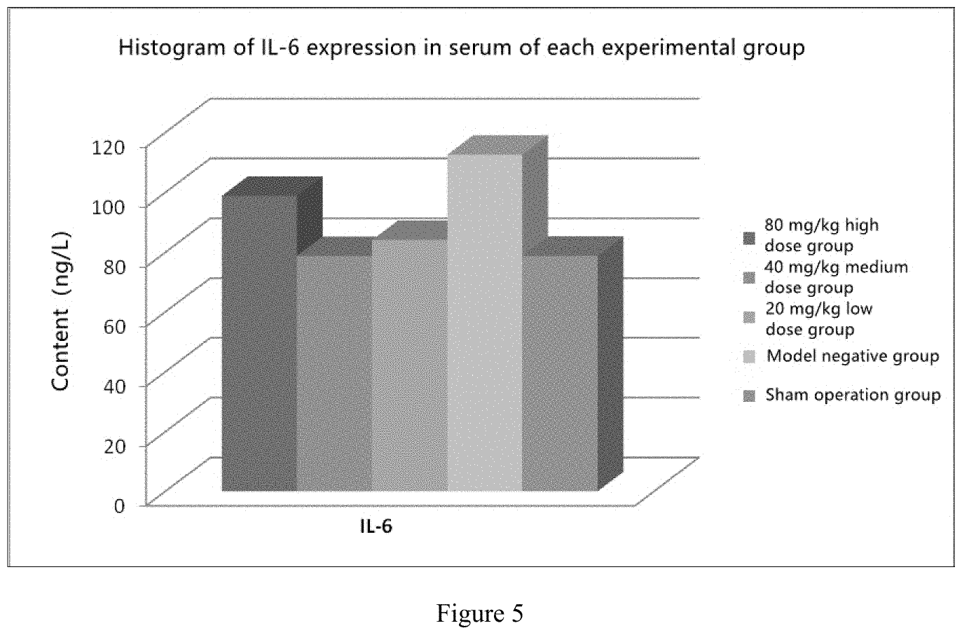

After 15 days of administration in each test group, the serum levels of TNF-α, IL-1β and IL-6 in 40 mg/kg and 20 mg/kg dose groups of chlorogenic acid were lower than those in the model-negative group, and the difference was significant (p<0.05), but for 80 mg/kg dose group, the difference was not significant. The results were shown in Table 3 and FIG. 3, FIG. 4, FIG. 5.

TABLE 3
The cytokine content in serum of rats in each experimental group

| | Dose | Cytokines (ng/L) | | |
|---|---|---|---|---|
| Groups | (mg/kg) | TNF-α | IL-1β | IL-6 |
| Chlorogenic acid | 80 | 54.48 ± 5.99# | 76.25 ± 4.62# | 98.82 ± 4.54# |
| high dose group | | | | |
| Chlorogenic acid | 40 | 35.25 ± 4.62* | 52.71 ± 6.39* | 78.73 ± 6.39* |
| middle dose group | | | | |
| Chlorogenic acid | 20 | 39.74 ± 6.04* | 58.54 ± 8.23* | 84.09 ± 5.16* |
| low dose group | | | | |
| Model negative | — | 64.32 ± 2.47# | 82.37 ± 5.88# | 112.56 ± 6.55# |
| group | | | | |
| Sham operation | — | 34.86 ± 4.39 | 54.43 ± 4.58 | 78.81 ± 8.32 |
| group | | | | |

Note:
*means p < 0.05 compared with the model negative group,
#means p < 0.05 compared with the sham operation group.

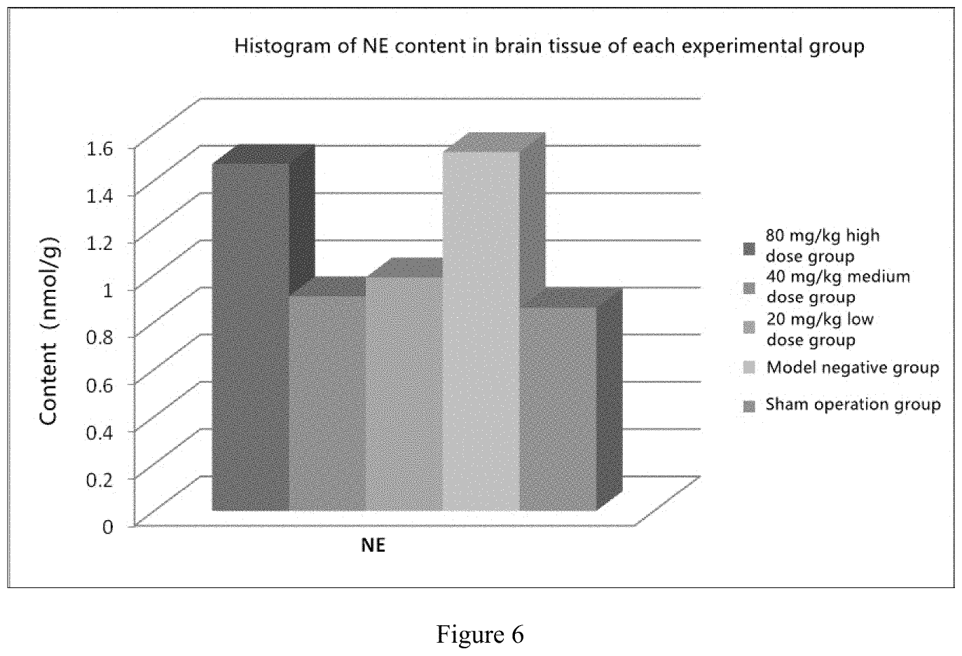

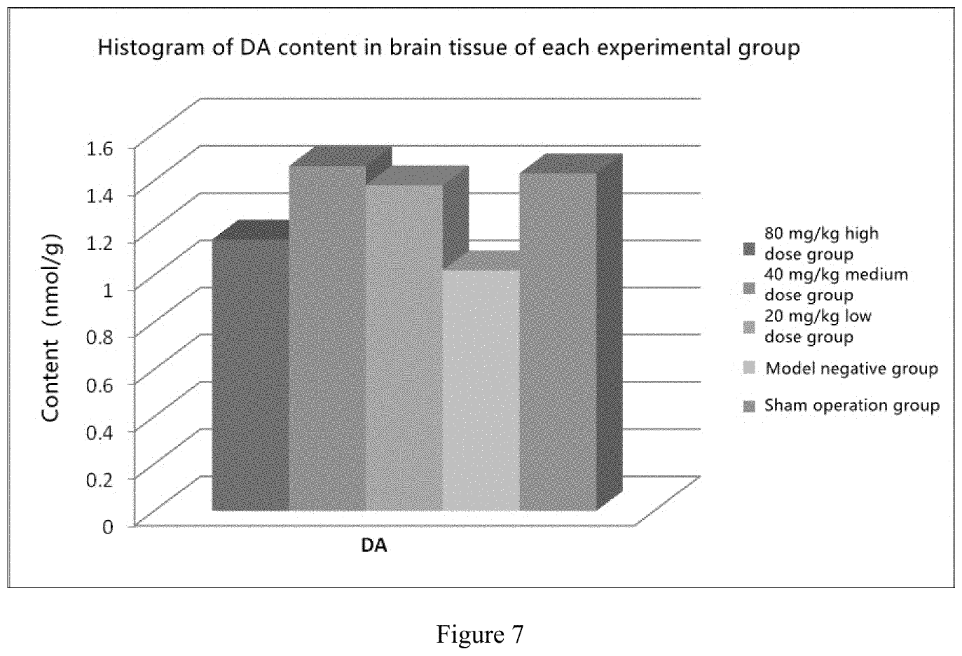

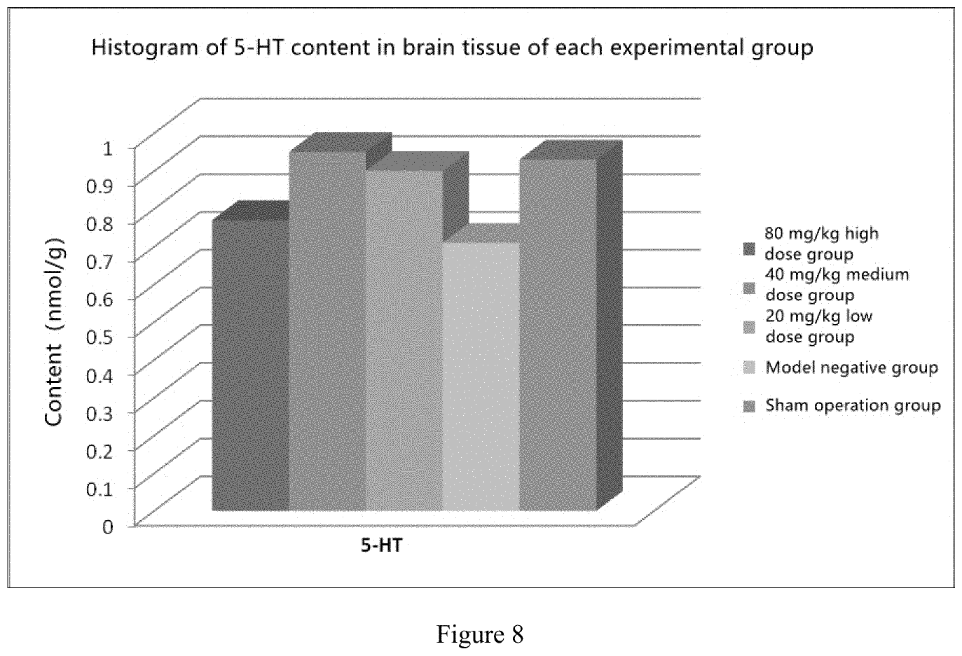

The contents of NE, DA, and 5-HT in the brain tissues of rats in 40 mg/kg and 20 mg/kg dose groups of chlorogenic acid were basically the same as those in the sham operation group, without significant difference (p>0.05), but compared with the model-negative group, the difference was significant (p<0.05). There was no significant difference between 80 mg/kg dose group and the model-negative group (p>0.05), while 80 mg/kg dose group had a significant difference from the sham operation group (p<0.05). The results were shown in Table 4 and FIG. 6, FIG. 7, and FIG. 8.

TABLE 4
The contents of NE, DA and 5-HT in the brain tissue of
rats in each test group (x̅ ± s)

| | Dose | Cytokines (nmol/g) | | |
|---|---|---|---|---|
| Groups | (mg/kg) | NE | DA | 5-HT |
| Chlorogenic acid | 80 | 1.47 ± 0.26# | 1.15 ± 0.12# | 0.77 ± 0.11# |
| high dose group | | | | |
| Chlorogenic acid | 40 | 0.91 ± 0.15* | 1.46 ± 0.08* | 0.95 ± 0.19* |
| middle dose group | | | | |
| Chlorogenic acid | 20 | 0.99 ± 0.18* | 1.38 ± 0.21* | 0.91 ± 0.15* |
| low dose group | | | | |
| Model negative | — | 1.52 ± 0.21# | 1.02 ± 0.26# | 0.71 ± 0.20# |
| group | | | | |
| Sham operation | — | 0.86 ± 0.23 | 1.43 ± 0.14 | 0.93 ± 0.09 |
| group | | | | |

Note:
*means p < 0.05 compared with the model negative group,
#means p < 0.05 compared with the sham operation group.

5. Summary

In each test group of the tibial implant model, among the indicators of mechanical hyperalgesia and radiant heat pain, 20-40 mg/kg dose groups of chlorogenic acid had significant differences compared with the model-negative group, in which 40 mg/kg dose group was equivalent with the sham operation group, indicating that chlorogenic acid could effectively improve and relieve cancer pain, and presented a dose-effect relationship within a certain dose range.

In the process of cancer pain, cytokines TNF-α, IL-1β, and IL-6 played important regulatory roles and were important regulatory mediators in the neuro-endocrine-immune function system. Noxious external stimuli would increase their expression, and their contents were directly related to the degree of cancer pain.

In each test group, the expression of TNF-α, IL-1β, and IL-6 detected in the serum of rats in the chlorogenic acid 20-40 mg/kg dose groups was significantly different from that of the model-negative group, and did not show significant difference from the sham operation group, indicating that chlorogenic acid could effectively regulate the serum levels of TNF-α, IL-1β, and IL-6, and maintain their normal levels. Thus, chlorogenic acid was expected to be able to improve and relieve cancer pain, that was consistent with the experimental results of pain behavior.

In brain tissue, NE, DA, and 5-HT were important participants in the cellular biological pathways related to pain perception. The contents of NE, DA, and 5-HT in the brain tissue of chlorogenic acid 20-40 mg/kg dose groups were basically same as those in the sham operation group, and there was no significant difference. However, compared with the negative model group, their contents were significant differences, indicating chlorogenic acid could induce the expression of DA and 5-HT caused by pain, and reduce the expression of NE. Thus, chlorogenic acid was shown to have the ability of improving and relieving pain, which was consistent with the experimental results of pain behavior.

In summary, chlorogenic acid could effectively ameliorate and alleviate pain, and could be used to prepare the drug or pharmaceutical composition of the present invention for preventing or treating pain. The drug of the present invention could effectively reduce mechanical hyperalgesia and radiant hyperalgesia caused by cancer, and had no toxic side effects. It could replace analgesic drugs such as opioids and non-steroidal anti-inflammatory drugs, and avoid the toxic and addictive effects due to long-term use of opioids, improve the life quality of patients, and have good clinical application prospects.

1. The use of chlorogenic acid in preparing drugs or pharmaceutical compositions for preventing or treating pain.

2. The use according to claim 1, characterized in that said pain is cancer pain, skeletal muscle pain, inflammatory pain, neuropathic pain, or immune pain;
Preferably, said cancer pain is neuropathic pain, nociceptive pain, or chronic pain;
Preferably, said skeletal muscle pain is bone joint pain; further preferably, the bone joint pain is knee joint pain, ankle joint pain, wrist joint pain, elbow joint pain, shoulder joint pain, patellar joint pain, hip joint pain, femoral joint pain, cervical and/or lumbar pain.

3. The use according to claim 1, characterized in that the prevention or treatment of pain is to reduce mechanical hyperalgesia or radiant hyperalgesia caused by cancer.

4. The use according to claim 1, characterized in that the drug or pharmaceutical composition is those inhibiting the expression of tumor necrosis factor α, interleukin 1β, and interleukin 6.

5. The use according to claim 1, characterized in that the drug or pharmaceutical composition is those regulating/increasing the expression of 5-hydroxytryptamine and dopamine caused by pain;
And/or the drug or pharmaceutical composition is those regulating/reducing the expression of noradrenaline caused by pain.

6. A pharmaceutical preparation for prevention or treatment of pain, comprising chlorogenic acid as an active ingredient and pharmaceutically acceptable excipients;
Preferably, the prevention or treatment of pain is to reduce mechanical hyperalgesia or radiant hyperalgesia caused by cancer.

7. The pharmaceutical preparation according to claim 6, characterized in that in the preparation, each preparation unit contains 1-3000 mg of chlorogenic acid; and the preparation unit is pill, tablet, packet, small ball, ampul or bottle.

8. The pharmaceutical preparation according to claim 7, characterized in that in the pharmaceutical preparation, the human dosage of chlorogenic acid is 1-10 mg/kg.

9. The pharmaceutical preparation according to claim 7, characterized in that the pharmaceutical preparations are oral preparations or injections.

10. A pharmaceutical composition for preventing or treating pain, characterized in that it is a pharmaceutical composition containing the pharmaceutical preparation according to claim 6.
